(19) United States
(12) Patent Application Publication
(10) Pub. No.: US 2021/0177267 A1
(43) Pub. Date: Jun. 17, 2021

(54) Component Concentration Measuring Device

(71) Applicant: Nippon Telegraph and Telephone Corporation, Tokyo (JP)

(72) Inventors: Yujiro Tanaka, Tokyo (JP); Masahito Nakamura, Tokyo (JP); Daichi Matsunaga, Tokyo (JP); Michiko Seyama, Tokyo (JP)

(21) Appl. No.: 17/051,543

(22) PCT Filed: Apr. 19, 2019
(86) PCT No.: PCT/JP2019/016807

§ 371 Date: Oct. 29, 2020

(30) Foreign Application Priority Data
May 1, 2018 (JP) 2018-088063

(51) Int. Cl. A61B 5/00; A61B 5/145

(52) U.S. Cl. CPC A61B 5/0095; A61B 5/14532

(57) ABSTRACT

A component concentration measurement device includes a light application unit that applies pulsed beam light of a wavelength that is absorbed by a target substance for measurement to a site of measurement, and a detection unit that detects a photoacoustic signal generated in the site of measurement where the beam light emitted from the light application unit has been applied. The light application unit applies the pulsed beam light with a pulse width at which a photoacoustic wave that occurs at a rising edge of a light pulse and a photoacoustic wave that occurs at a falling edge of the light pulse do not interfere with each other.

### CROSS-REFERENCE TO RELATED APPLICATIONS

This application is a national phase entry of PCT Application No. PCT/JP2019/016807, filed on Apr. 19, 2019, which claims priority to Japanese Application No. 2018-088063, filed on May 1, 2018, which applications are hereby incorporated herein by reference.

### TECHNICAL FIELD

The present invention relates to a component concentration measurement device for non-invasively measuring glucose concentration.

### BACKGROUND

In terms of determining a dose of insulin for a diabetes patient or preventing diabetes, it is important to know (measure) blood sugar level. The blood sugar level is the concentration of glucose in blood, and as a way of measuring this kind of component concentration, a photoacoustic method is well known (see Non-Patent Literatures 1, 2 and 3).

When a certain amount of light (an electromagnetic wave) is applied to a living body, the applied light is absorbed by molecules contained in the living body. As a result, target molecules for measurement in a portion applied with the light are locally heated to expand and generate a sound wave. The pressure of the sound wave depends on the amount of molecules that absorb the light. The photoacoustic method measures this sound wave to measure the amount of molecules in the living body. A sound wave is a pressure wave that propagates within a living body and has a property of being resistant to scattering compared to an electromagnetic wave; the photoacoustic method can be regarded to be a suitable way for measuring blood components in a living body.

Measurement by the photoacoustic method enables continuous monitoring of the glucose concentration in blood. In addition, measurement with the photoacoustic method does not require blood sample and causes no discomfort in a subject of measurement.

### CITATION LIST

### Patent Literature

Patent Literature 1: Japanese Patent Laid-Open No. 2010-104858

### Non-Patent Literature

Non-Patent Literature 1: P. P. Pai et al., “A Photoacoustics based Continuous Non-Invasive Blood Glucose Monitoring System”, Medical Measurements and Applications Proceedings, vol. 15278142, pp. 1-5, 2015.
Non-Patent Literature 2: J. Laufer et al., “In vitro measurements of absolute blood oxygen saturation using pulsed near-infrared photoacoustic spectroscopy: accuracy and resolution”, Physics in Medicine and Biology, vol. 50, pp. 4409-4428, \\\\.

### SUMMARY

### Technical Problem

For continuous measurement of the glucose concentration in blood by the photoacoustic method mentioned above, downsizing of the device is important. Meanwhile, for achieving sufficient measurement sensitivity, it is important to apply light of high energy within a possible range to produce a large sound wave. Application of high-energy light, however, requires a large light source and the like, which hampers downsizing. In a measurement by the photoacoustic method, pulsed beam light is applied to a site of measurement; and it is conceivable to increase the pulse width of the beam light to thereby increase the applied light energy in conjunction with downsizing.

However, measurements with increased light energy by the expansion of the pulse width encountered a problem of the intensity of a photoacoustic wave not changing linearly with respect to change in light intensity. Under such a condition, accurate measurement cannot be performed.

In order to solve this drawback, an object of embodiments of the present invention is to allow sufficient measurement sensitivity to be achieved in measurements by the photoacoustic method even on a downsized device without reducing the measurement accuracy.

### Means for Solving the Problem

A component concentration measurement device according to embodiments of the present invention includes: a light application unit that applies pulsed beam light of a wavelength that is absorbed by a target substance for measurement to a site of measurement; and a detection unit that detects a photoacoustic signal generated in the site of measurement where the beam light emitted from the light application unit has been applied, wherein the light application unit applies the pulsed beam light with a pulse width at which a photoacoustic wave that occurs at a rising edge of a light pulse and a photoacoustic wave that occurs at a falling edge of the light pulse do not interfere with each other.

In the component concentration measurement device, the light application unit may apply the pulsed beam light with a pulse width of a duration for which the photoacoustic wave that occurs at the rising edge of the light pulse lasts.

In the component concentration measurement device, the substance is glucose, and the light application unit applies the beam light of a wavelength that is absorbed by glucose. In this case, the light application unit may apply the beam light with a pulse width of 0.02 seconds or longer.

### Effects of Embodiments of the Invention

As described above, embodiments of the present invention are configured to apply pulsed beam light with a pulse width at which a photoacoustic wave that occurs at the rising edge of a light pulse and a photoacoustic wave that occurs at the falling edge of the light pulse do not interfere with each other. Thus, it provides an advantageous effect of allowing sufficient measurement sensitivity to be achieved in measurements by the photoacoustic method without reducing S/N even on a downsized device.

### BRIEF DESCRIPTION OF THE DRAWINGS

FIG. 1 is configuration diagram showing a configuration of a component concentration measurement device in an embodiment of the present invention.

FIG. 2 is a characteristics diagram showing the states of photoacoustic waves (a) when a photoacoustic wave that occurs at a rising edge of a light pulse interferes with a photoacoustic wave that occurs at a falling edge of the light pulse, and (b) when they do not interfere with each other.

FIG. 3 is configuration diagram showing a more detailed configuration of the component concentration measurement device in an embodiment of the present invention.

### DETAILED DESCRIPTION OF ILLUSTRATIVE EMBODIMENTS

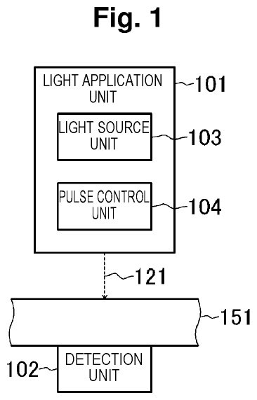

Referring to FIG. 1, a component concentration measurement device in an embodiment of the present invention is described. The component concentration measurement device includes a light application unit 101 that applies pulsed beam light 121 of a wavelength that is absorbed by a target substance for measurement to a site of measurement 151, and a detection unit 102 that detects a photoacoustic signal. The detection unit 102 detects a photoacoustic signal generated in the site of measurement 151 where the beam light 121 emitted from the light application unit 101 has been applied. The beam light 121 has a beam diameter of about 100 μm.

In this embodiment, the light application unit 101 applies the pulsed beam light 121 with a pulse width at which a photoacoustic wave that occurs at a rising edge of a light pulse and a photoacoustic wave that occurs at a falling edge of the light pulse do not interfere with each other. For example, the light application unit 101 applies the pulsed beam light with a pulse width of a duration for which the photoacoustic wave that occurs at the rising edge (or the falling edge) of the light pulse lasts.

For example, when the target substance for measurement is glucose in blood, the light application unit 101 includes a light source unit 103 that generates beam light 121 of a wavelength that is absorbed by glucose, and a pulse control unit 104 that turns the beam light 121 generated by the light source into pulsed light of a set pulse width. Glucose exhibits absorbency in light wavelength bands around 1.6 μm and around 2.1 μm (see Patent Literature 1). The pulse control unit 104 creates the pulsed beam light 121 mentioned above. When glucose is the target substance, the light application unit 101 (the pulse control unit 104) applies beam light 121 with a pulse width of 0.02 seconds or longer.

For measurements of this kind, the inventors have discovered the phenomenon of the intensity of the photoacoustic wave not changing linearly with respect to change in light intensity when conducting measurements with increased light energy by expanding the pulse width of the beam light being applied. After intensive study on this phenomenon, the inventors found the following: application of a light pulse in measurement causes acoustic waves to occur at both the rising edge and falling edge of the light pulse, and depending on the pulse width, these acoustic waves interfere with each other, preventing measurement of photoacoustic intensity that linearly corresponds to the intensity of the light applied.

Intensive study based on the foregoing findings by the inventors has led to embodiments of the present invention, which can suppress reduction in the accuracy of photoacoustic signals by setting the pulse width of the beam light to be applied in a range that prevents interference between a photoacoustic wave that occurs at the rising edge of a light pulse and a photoacoustic wave that occurs at the falling edge of the light pulse. By expanding the pulse width of beam light under such a condition, it is possible to enhance the energy of the beam light being applied to obtain sufficient measurement sensitivity without reducing the measurement accuracy.

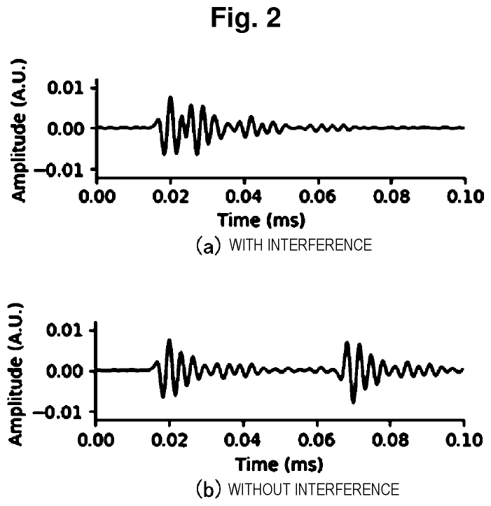

For example, in a case where a photoacoustic wave that occurs at the rising edge of a light pulse interferes with a photoacoustic wave that occurs at the falling edge of the light pulse, a photoacoustic wave would be measured as shown in FIG. 2(a). In this situation, the peaks of the waveform may not correctly correspond to the component concentration. In contrast, when the pulse width is appropriately set to create a state in which the photoacoustic wave that occurs at the rising edge of a light pulse and the photoacoustic wave that occurs at the falling edge of the light pulse do not interfere with each other, a photoacoustic wave in which individual peaks clearly appear is measured as shown in FIG. 2(a). In this situation, the peaks of the waveform correctly correspond to the component concentration, enabling accurate measurements.

For example, when glucose is to be measured, the absorption length will be in a near-infrared region (1100-1800 nm). In this case, the photoacoustic wave that is generated by the application of beam light (that occurs at the rising edge of a light pulse) lasts for about 0.02 s. Thus, in order to avoid interference between the photoacoustic wave that occurs at the rising edge of the light pulse and the photoacoustic wave that occurs at the falling edge of the light pulse, the beam light should be applied at a pulse width of 0.02 s or longer.

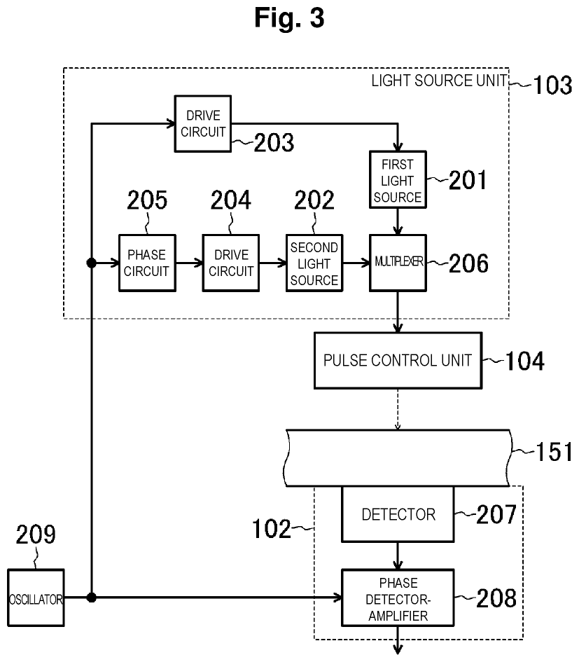

Now referring to FIG. 3, the component concentration measurement device is described in more detail. The component concentration measurement device includes a first light source 201, a second light source 202, a drive circuit 203, a drive circuit 204, a phase circuit 205, a multiplexer 206, a detector 207, a phase detector-amplifier 208, and an oscillator 209. The first light source 201, the second light source 202, the drive circuit 203, the drive circuit 204, the phase circuit 205, and the multiplexer 206 constitute the light source unit 103. The detector 207 and the phase detector-amplifier 208 constitute the detection unit 102.

The oscillator 209 is connected to each of the drive circuit 203, the phase circuit 205, and the phase detector-amplifier 208 via signal wires. The oscillator 209 sends a signal to each of the drive circuit 203, the phase circuit 205, and the phase detector-amplifier 208.

The drive circuit 203 receives the signal sent from the oscillator 209, and supplies driving electric power to the first light source 201, which is connected by a signal wire, to cause the first light source 201 to emit light. The first light source 201 is a semiconductor laser, for example.

The phase circuit 205 receives the signal sent from the oscillator 209, and sends a signal generated by giving a phase shift of 180° to the received signal to the drive circuit 204, which is connected by a signal wire.

The drive circuit 204 receives the signal sent from the phase circuit 205, and supplies driving electric power to the second light source 202, which is connected by a signal wire, to cause the second light source 202 to emit light. The second light source 202 is a semiconductor laser, for example.

The first light source 201 and the second light source 202 output light of different wavelengths from each other and direct their respective output light to the multiplexer 206 via light wave transmission means. For the first light source 201 and the second light source 202, the wavelength of light of one of them is set to a wavelength that is absorbed by glucose, while the wavelength of light of the other is set to a wavelength that is absorbed by water. Their respective wavelengths are also set such that degrees of their absorption will be equivalent.

The light output by the first light source 201 and the light output by the second light source 202 are multiplexed in the multiplexer 206 and are incident onto the pulse control unit 104 as one light beam. Upon incidence of the light beam, the pulse control unit 104 applies the incident light beam to the site of measurement 151 as pulsed light of a predetermined pulse width. Inside the site of measurement 151 thus applied with the pulsed light beam, a photoacoustic signal is generated.

The detector 207 detects the photoacoustic signal generated in the site of measurement 151, converts it into an electric signal, and sends it to the phase detector-amplifier 208, which is connected by a signal wire. The phase detector-amplifier 208 receives a synchronization signal necessary for synchronous detection sent from the oscillator 209, and also receives the electric signal proportional to the photoacoustic signal being sent from the detector 207, performs synchronous detection, amplification and filtering on it, and outputs an electric signal proportional to the photoacoustic signal.

The first light source 201 outputs light that has been intensity-modulated in synchronization with an oscillation frequency of the oscillator 209. In contrast, the second light source 202 outputs light that has been intensity-modulated with the oscillation frequency of the oscillator 209 and in synchronization with the signal that has gone through a phase shift of 180° in the phase circuit 205.

The intensity of the signal output by the phase detector-amplifier 208 is proportional to the amounts of components in the site of measurement 151. This is because the light that is output by each of the first light source 201 and the second light source 202 is proportional to the amount of light that was absorbed by the components (glucose, water) in the site of measurement 151. From a measured value of the strength of the signal thus output, a component concentration derivation unit (not shown) determines the amount of the target component (glucose) in blood at the site of measurement 151.

As mentioned above, the light output by the first light source 201 and the light output by the second light source 202 have been intensity-modulated with signals of the same frequency. Accordingly, there is no effect of unevenness in frequency characteristics of a measurement system, which is problematic in the case of intensity modulation with signals of multiple frequencies.

Meanwhile, non-linear dependence on absorption coefficient that exists in measured values of photoacoustic signals, which is problematic in measurements by the photoacoustic method, can be solved by performing measurements using light of multiple wavelengths that gives an equal absorption coefficient as described above (see Patent Literature 1).

As described above, embodiments of the present invention are configured to apply pulsed beam light with a pulse width at which a photoacoustic wave that occurs at the rising edge of a light pulse and a photoacoustic wave that occurs at the falling edge of the light pulse do not interfere with each other. This allows sufficient measurement sensitivity to be achieved in measurements by the photoacoustic method without reducing S/N even on a downsized device.

It will be apparent that the present invention is not limited to the above-described embodiments but many variations and combinations may be made by ordinarily skilled persons in the art within the technical idea of the invention.

### REFERENCE SIGNS LIST

101 light application unit
102 detection unit
103 light source unit
104 pulse control unit
121 beam light
151 site of measurement.

1.-4. (canceled)

5. A component concentration measurement device comprising:
a light applicator that applies, to a site of measurement, pulsed beam light of a wavelength that is absorbed by a target substance for measurement; and
a detector that detects a photoacoustic signal generated in the site of measurement where the pulsed beam light emitted from the light applicator has been applied, wherein the light applicator applies the pulsed beam light with a first pulse width, and wherein the first pulse width is a pulse width at which a first photoacoustic wave that occurs at a rising edge of a light pulse and a second photoacoustic wave that occurs at a falling edge of the light pulse do not interfere with each other.

6. The component concentration measurement device according to claim 5, wherein the light applicator applies the pulsed beam light with the first pulse width for a duration of the first photoacoustic wave that occurs at the rising edge of the light pulse.

7. The component concentration measurement device according to claim 5, wherein:
the target substance is glucose; and
the wavelength of the pulsed beam light is a wavelength that is absorbed by glucose.

8. The component concentration measurement device according to claim 7, wherein the first pulse width is 0.02 seconds or longer.

9. A method comprising:
applying, to a site of measurement, pulsed beam light of a wavelength that is absorbed by a target substance for measurement; and
detecting a photoacoustic signal generated in the site of measurement where the pulsed beam light emitted has been applied, wherein the pulsed beam light has a first pulse width, and wherein the first pulse width is a pulse width at which a first photoacoustic wave that occurs at a rising edge of a light pulse and a second photoacoustic wave that occurs at a falling edge of the light pulse do not interfere with each other.

10. The method according to claim 9, wherein the pulsed beam light is applied for a duration of the first photoacoustic wave that occurs at the rising edge of the light pulse.

11. The method according to claim 9, wherein:
the target substance is glucose; and
the wavelength of the pulsed beam light is a wavelength that is absorbed by glucose.

12. The method according to claim 11, wherein the first pulse width is 0.02 seconds or longer.
